(19) United States
(12) Patent Application Publication
(10) Pub. No.: US 2005/0096752 A1
(43) Pub. Date: May 5, 2005

(54) Biomedical device

(76) Inventors: Kevin Warwick, Reading (GB); Brian Andrews, Aylesbury HP21 8AL (GB)

Correspondence Address: RICK MARTIN;PATENT LAW OFFICES OF RICK MARTIN, PC, 416 COFFMAN STREET, LONGMONT, CO, 80501, US

(21) Appl. No.: 10/505,651

(22) PCT Filed: Feb. 24, 2003
(86) PCT No.: PCT/GB03/00798

(30) Foreign Application Priority Data
Feb. 23, 2002 (GB) 0204288.5

(51) Int. Cl. A61F 2/70; A61B 5/04

(52) U.S. Cl. 623/25; 623/24

(57) ABSTRACT

A device to be wholly or partly implanted into a patient's body comprises: one or more sensors to be placed at any appropriate location of said body and adapted to obtain, in use, one or more sensory and/or motor signals from the body's nervous system and/or from the pressure or temperature exerted on the body; one or more electrodes to be placed at any appropriate location of said body and adapted to feed, in use, sensory and/or motor signals to the body's nervous system; and a control means incorporating a memory of one or more predetermined signals, a comparator to compare the sensory and/or motor signals obtained with said predetermined signals, and means for producing sensory and/or motor signals to feed any appropriate location of the body's nervous system; the magnitude of these latter sensory and/or motor signals being dependent on the outcome of the comparison conducted by the comparator.

### FIELD OF THE INVENTION

The invention relates to devices which are wholly or partly implanted into a patient's body and particularly those used in the fields of biomedical engineering, neuroscience and cybernetics. More particularly, the device of this invention is intended to allow useful connections with the nervous system to be made.

The present invention aims to increase the function in people with injuries or malfunctions such as spinal cord injury, limb amputation, quadriplegia, bowel and bladder malfunction etc.

Another object of the present invention is to provide a system which can be integrated to artificial limbs to enable a patient to feel sensations similar to those of the amputated limb and allow more precise control of powered prostheses.

A further object of this invention is to provide a device which can be fully implanted into a patient's body.

The invention envisages that the part of the device which is implanted into a patient's body is controlled via a wireless link from an external computer.

It is also an objective of this invention to provide a relatively low cost and minimally invasive device.

The invention aims to enable neural prostheses to restore sensory and motor functions lost by spinal injury, other neurological lesions or limb amputation.

### SUMMARY OF THE INVENTION

According to the first broad independent aspect of the invention there is provided a device to be wholly or partly implanted into a patient's body and comprising:
One or more sensors to be placed at any appropriate location of said body and adapted to obtain, in use, one or more sensory and/or motor signals from the body's nervous system and/or from the pressure or temperature exerted on the body;
One or more sensors to be placed at any appropriate location of said body and adapted to feed, in use, sensory and/or motor signals to the body's nervous system; and
A control means incorporating a memory of one or more predetermined signals, a comparator to compare the sensory and/or motor signals obtained with said predetermined signals, and means for producing sensory and/or motor signals to feed any appropriate location of the body's nervous system;
The magnitude of these latter sensory and/or motor signals being dependent on the outcome of the comparison conducted by the comparator.

This device is advantageous because it can provide control of the transmission of sensory and motor signals and by so doing assist the body's nervous system, modify its function for any given reason or even substitute any appropriate part of the body's nervous system.

In a subsidiary aspect of the present invention, the control means is adapted to feed one or more motor signals to cause, in use, the contraction or expansion of one or more muscles of the body and to obtain one or more sensory signals during the contraction or expansion of one or more of said muscles.

These subsidiary features are particularly advantageous because they can permit the specific control of the contraction or expansion of muscles so as to prevent the excessive contraction or expansion of the muscles and to avoid the possible consequential damage of an excessive contraction or expansion of these muscles.

In a further subsidiary aspect of the invention, the sensors are constituted by one array of microelectrodes to be located upstream from a lesion operating in conjunction with another array of microelectrodes downstream from said lesion.

This is particularly advantageous because it will facilitate the transmission of nervous signals across the lesion and will lead to an improved motion and sensitivity.

In one particularly advantageous format, each array comprises approximately one hundred microelectrodes.

This number has been chosen because, these will be sufficient to generate and transmit a useful amount of sensory and motor signals to the body's nervous system.

According to a further advantageous aspect of the invention, the sensors are microelectrodes, the or each tip of which is adapted to be inserted in use into the or each nerve trunk.

This is particularly advantageous because at these locations, the control system can obtain and transmit useful signals to achieve its motor and/or sensory objectives.

According to a second broad independent aspect, there are provided means enabling the flexure of a patient's body member, comprising:
a) an array of microelectrodes implanted into the or each nerve portion,
b) means producing one or more motor signals sufficient to cause the stretching or contraction of one or more muscles,
c) means for obtaining one or more sensory signals from one or more microelectrodes during the stretching or contraction of one or more muscles,
d) means for comparing the or each obtained sensory signals with a set of predetermined sensory signals,
e) means for producing one or more motor signals to adjust the stretching or contraction of one or more muscles in accordance with the outcome of the comparison carried out in (d).

This is particularly advantageous because it provides the means for stretching or contracting muscles, while at the same time avoiding excessive stretching or contraction which could lead to damage to any part of the body or the object of the stretching or contraction.

### EMBODIMENTS OF THE PRESENT INVENTION

a) Improved Hand Function

The median nerve contains a mixture of many individual sensory and motor axons. The sensory axons conduct signals generated by skin receptors in response to temperature and pressure changes applied in the region of the thumb, index and middle fingers and palm. Motor axons are located within the median nerve and conduct signals from the spinal cord to muscles such as the thenar muscles group located at the base of the thumb.

In this embodiment, the array of microelectrodes is inserted into the median nerve such that the sensitive tips of the microelectrodes are distributed within the nerve trunk. In this configuration, some electrodes can pick up signals from sensory axons whilst others pick up mainly motor axon signals whilst others pick up a mix of the two.

The array is connected to a control means incorporating an external amplifier and a signal processing system, through fine wires passing through the skin.

The microelectrodes and the fine wires will be chosen by the person skilled in the art and adapted to cause minimal discomfort for a prolonged period without infection.

The control means of this embodiment comprises recorder means or a memory adapted to register nerve signals from carefully selected individual axons. The person skilled in the art will also be able to discriminate through empirical research background noise from the individual nerve signals. He will also conduct a series of tests to identify specific sensory stimuli for a variety of points on the skin while using the memory of the control system to record these microelectrode signals. These tests can be conducted for a variety of specific sensory stimuli such as touch, vibration, heat and light.

Similar tests can be carried out with regard to motor signals, in this case a patient will contract a specific muscle to generate controlled movement and force whilst the corresponding activity from the microelectrodes is recorded.

In both of the previous tests, the control means can be used to determine the motor and sensory activity and even to separate out the motor signals from the sensory signals.

In this embodiment, it is also foreseen that the control means will be used to apply low level electrical signals to various microelectrodes in the array. In this case, when a motor signal or stimulus is applied to motor axons the corresponding muscle fibres will contract. When an appropriate stimulus is applied to sensory axons these may be perceived by the patient as sensations.

Through these various experiments, the memory of the control means will be provided with patterns of low level electrical stimulation destined to synthesise sensations or motor signals.

Each microelectrode will be adapted to transmit and receive two-way signals between nerves and external microcomputer means by wires through the skin. The invention also perceives through the skin wires being replaced by a wireless radio frequency link connecting the fully implanted component with the external control computers or control means.

The device of the present invention can even be used to facilitate the movement and the feel of the hands of quadriplegics. In this situation, microelectrode arrays are inserted into the median and radial nerves. The muscles that control the hand are activated using electrical pulses to microelectrodes close to the axons enervating those muscles. The control means is used to precisely generate the required electrical pulses so that in effect the combination of the control means and the microelectrodes forms a neuroprosthesis. Receptors in the patient's skin and muscle will fire as the hand opens, makes a contact and grasps an object. The receptor signals is then detected by the microelectrodes positioned close to the appropriate axons and fed out to the controlling microcomputer or control means which in turn would automatically regulate using comparator means the degree of activation of muscles so as to cause the hand to grip in the most appropriate fashion. Obtaining one or more sensory signals during the contraction or expansion of the muscles will enable the control means to specifically regulate the degree of contraction or expansion as the hand grasps an object while avoiding damage to the object or to the hand and its associated elements.

b) Spinal Injury

After a spinal injury the nervous tissue below the lesion is usually alive and operating even though it is disconnected from the brain i.e. signals are still being naturally generated by sensory receptors and transmitted to the spinal cord but are not perceived by the brain. Similarly signals are still being put out by the spinal cord and causing muscles to contract.

In this embodiment, typically two arrays are used, one to pick up sensory signals from a paralysed nerve and then another being mapped into a nerve containing sensory axons connecting with the spinal cord above the lesion level. For example in paraplegia, signals from the array in the posterior femoral cutaneous nerve can be mapped onto a nerve eminating from the thoracic spine.

c) Improved Bladder and Bowel Function

The array would allow selective stimulation of the detrusor and sphincter. In the bowel selective stimulation provided by the invention's control means will allow high frequency fatigue of the sphincter with increased colonic activity whilst the abdominals will provide increased intra abdominal pressure to expel the faeces.

d) Improved Leg Function

Arrays in the femoral or sciatic nerves, or branches thereof, will allow more precise and fatigue-free lower limb function as well as provide sensory signals for control and biofeedback.

e) Improved Artificial Limbs

Amputees still have living nerves in their stumps into which microelectrode arrays can be inserted. These nerve stumps still relay voluntary signals to amputated muscles and are still capable of conducting sensory signals that previously originated in the amputated skin and muscles. For these amputees standard force, pressure and temperature sensors can be built into the artificial limb.

These sensors could be connected to a control means which would in turn generate and apply forces or signals to electrode tips that had been previously associated with the appropriate sensation.

If for example a hand amputee wearing such prostheses fitted with miniature pressure sensors in the index finger tip were to touch or press an object, the finger tip sensor would generate an electrical signal proportional to the applied pressure. This pressure signal can then by the device of the present invention be utilised to enable the application of appropriate stimulus pulses to sensory nerve fibres within the stump using a microelectrode array to recreate realistic sensation of the pressure at the index finger tip.

1-7. (canceled)

8. A device to be wholly or partly implanted into a patient's body, said device comprising:
one or more sensors to be placed at any appropriate location of said body and adapted to obtain, in use, one or more sensory and/or motor signals from the body's nervous system and/or from pressure or temperature exerted on the body;
one or more sensors to be placed at any appropriate location of said body and adapted to feed, in use, sensory and/or motor signals to the body's nervous system;
and a control means incorporating a memory of one or more predetermined signals;
a comparator to compare the sensory and/or motor signals obtained with said predetermined signals, and means for producing sensory and/or motor signals to feed any appropriate location of the body's nervous system; the magnitude of these latter sensory and/or motor signals being dependent on the outcome of the comparison conducted by the comparator.

9. A device according to claim 8 wherein the control means is adapted to feed one or motor signals to cause, in use, the contraction or expansion of one or more muscles of the body and to obtain one or more sensory signals during the contraction or expansion of one or more of said muscles.

10. A device according to claim 8 wherein the sensors are constituted by one array of microelectrodes to be located upstream from a lesion operating in conjunction with another array of microelectrodes downstream from said lesion.

11. A device according to claim 9 wherein the sensors are constituted by one array of microelectrodes to be located upstream from a lesion operating in conjunction with another array of microelectrodes downstream from said lesion.

12. A device according to claim 10 wherein each array comprises approximately 100 microelectrodes.

13. A device according to claim 11 wherein each array comprises approximately 100 microelectrodes.

14. A device according to claim 8, wherein the sensors are microelectrodes, the or each tip of which is adapted to be inserted in use into the or each nerve trunk.

15. Means enabling the flexure of a patient's body member, comprising:
(a) an array of microelectrodes implanted into one or more nerve portion,
(b) means producing one or more motor signals sufficient to cause the stretching or contraction of one or more muscles,
(c) means for obtaining one or more sensory signals from one or more microelectrodes during the stretching or contraction of one or more muscles,
(d) means for comparing the or each obtained sensory signals with a set of predetermined sensory signals,
(e) means for producing one or more motor signals to adjust the stretching or contraction of one or more muscles in accordance with the outcome of the comparison carried out in (d).
